(19) United States
(12) Patent Application Publication
(10) Pub. No.: US 2004/0052752 A1
(43) Pub. Date: Mar. 18, 2004

(54) Cosmetic composition containing a block copolymer consisting of blocks with different refractive indices

(76) Inventors: Henri Samain, Bievres (FR); Nathalie Mougin, Paris (FR); Christophe Dauga, Levallois-Peter (FR)

Correspondence Address: D Douglas Price, Steptoe & Johnson, 1330 Connecticut Avenue NW, Washington, DC, 20036, US

(21) Appl. No.: 10/333,228

(22) PCT Filed: Jul. 11, 2001
(86) PCT No.: PCT/FR01/02244

(30) Foreign Application Priority Data
Jul. 18, 2000 (FR) 0009405

(51) Int. Cl. A61K 7/06; A61K 7/11

(52) U.S. Cl. 424/70.16

(57) ABSTRACT

The invention concerns a cosmetic composition comprising, in a cosmetically acceptable medium, at least a block copolymer consisting of at least two polymer blocks, each of the polymer blocks having a refractive index more or less by at least 0.1 unit than the refractive index of the neighbouring polymer block(s). Said composition can be used for hairstyling.

The present invention relates to a cosmetic composition comprising at least one block copolymer with particular optical properties and to its application in treating the hair, especially in shaping hair.

In cosmetics, styling products are especially used to increase the volume of the hairstyle, to shape the hair and to hold the hairstyle. The current products effect little or no impairment of the sheen of the hair, but they do not improve it either.

The Applicant has found, suprisingly, that by using compositions comprising, in a cosmetically acceptable medium, block copolymers with particular optical properties, the sheen can be appreciably improved while at the same time maintaining the natural appearance of the hair.

Moreover, it has also been found that the use of these compositions gives a sensation of hair that feels thicker.

One subject of the invention is thus a cosmetic composition comprising, in a cosmetically acceptable medium, a block copolymer consisting of at least two polymer blocks, each of the polymer blocks having a refractive index that is at least 0.1 unit greater or less than the refractive index of the neighbouring block(s).

A subject of the invention is also a process for treating the hair.

Another subject of the invention consists of a process for shaping the hair.

Other subjects, characteristics, aspects and advantages of the invention will emerge even more clearly on reading the description and the various examples that follow.

The cosmetic composition in accordance with the invention comprises, in a cosmetically acceptable medium, at least one block copolymer consisting of at least two polymer blocks A and B, each of the polymer blocks A having a refractive index that is at least 0.1 unit greater or less than the refractive index of the neighbouring polymer block(s) B.

The refractive index is measured on samples, in the form of films, of a polymer consisting of the monomer(s) of a block. This polymer film is formed by coating onto a silicon wafer and the refractive index measurement is performed by phase-modulated ellipsometry on a Jobin Yvon Uvisel DH10 machine. The measuring conditions are as follows:

angle of incidence: 70°

beam diameter: 1 mm

integration time: 200 ms.

The machine makes it possible to determine the thickness of the oxide layer which has been able to form naturally in air on the silicon wafer, the thickness of the polymer and its refractive index for wavelengths from 248 to 827 nm and preferably from 400 to 700 nm.

Each block of the block copolymer is thus characterized by a refractive index. If the refractive index of a block A of the block copolymer is denoted by n_{A} and the refractive index of the adjacent block B of the block copolymer is denoted by n_{B}, these two indices must satisfy the following condition:

|n_{A}-n_{B}|≧0.1.

The number of blocks present in the block copolymer of the invention is greater than or equal to 2 and is preferably equal to 2 or 3.

The block copolymers that are suitable in the invention are synthesized from monomers chosen especially from acrylic acid, methacrylic acid, N,N-dimethylacrylamide, quaternized or unquaternized dimethylaminoethyl methacrylate, methacrylamide, N-t-butylacrylamide, maleic acid and its hemiesters, maleic anhydride, crotonic acid, itaconic acid, acrylamide, hydroxylated (meth)acrylates such as hydroxyethyl methacrylate, diallyldimethylammonium chloride, vinylpyrrolidone, vinyl ethers such as methyl vinyl ether, maleimides, vinylpyridine, vinylimidazole, other polar heterocyclic vinyl compounds, styrenesulfonates, allylic alcohols, vinyl alcohols, salts of any of the acids and amines listed above, acrylic or methacrylic acid esters of C₁-C₁₃ alcohols, for instance methanol, ethanol, methoxyethanol, 1-propanol, 2-propanol, 1-butanol, 2-methyl-1-propanol, 1-pentanol, 2-pentanol, 3-pentanol, 2-methyl-1-butanol, 1-methyl-1-butanol, 3-methyl-1-butanol, 1-methyl-1-pentanol, 2-methyl-1-pentanol, 3-methyl-1-pentanol, t-butanol, cyclo-hexanol, neodecanol, 2-ethyl-1-butanol, 3-heptanol, benzyl alcohol, 2-octanol, 6-methyl-1-heptanol, 2-ethyl-1-hexanol, 3,5-dimethyl-1-hexanol, 3,5,5-trimethyl-1-hexanol, 1-decanol, 1-dodecanol, 1-hexadecanol, 1-octadecanol, and other similar alcohols, preferably C₁-C₁₂ alcohols, fluoroacrylates such as ethyl 2-perfluorooctylacrylate, styrene, a polystyrene, vinyl acetate, vinyl chloride, vinylidene chloride, vinyl propionate, α-methylstyrene, t-butyl-styrene, butadiene, cyclohexadiene, ethylene, propylene, vinyltoluene, and mixtures thereof.

The above monomers may be polymerized by any polymerization technique known to those skilled in the art, for example by free-radical, anionic or cationic polymerization, or by polycondensation. A controlled free-radical polymerization is preferably used. This recent technique is especially described in “New Method of Polymer Synthesis”, Blackie Academic & Professional, London, 1995, Volume 2, page 1, or Trends Polym. Sci. 4, page 183 (1996) by C. J. Hawker), and in particular JACS, 117, page 5614 (1995) by Matyjasezwski et al. describes the free-radical polymerization by atom transfer. These techniques now make it possible to perform the free-radical synthesis of a very wide variety of block copolymers “to order” under working conditions that are more readily industrializable than was the case for anionic or cationic polymerization, and thus allows an adjustment of the physicochemical properties of the polymers as a function of the intended application.

The copolymer thus polymerized, which is preferably used in the cosmetic composition of the invention, is a polystyrene/poly(ethyl 2-perfluorooctylacrylate).

The block copolymers according to the invention have a weight-average molar mass, measured by light scattering, of between 10 000 and 500 000 g/mol and preferably between 20 000 and 200 000 g/mol.

The proportion of block copolymer(s) is between 0.001% and 10% by weight and preferably between 0.1% and 5% by weight relative to the total weight of the cosmetic composition.

The cosmetic compositions according to the invention contain a cosmetically acceptable medium in which the block copolymer of the invention may be soluble or dispersed. This medium comprises water and/or cosmetically acceptable organic solvents.

The expressions “cosmetically acceptable medium” and “cosmetically acceptable solvent” mean a medium and a solvent that are compatible with any keratin material, such as the skin, the nails, the hair, the eyelashes, the eyebrows, the lips and any other area of the body and the face.

The organic solvents may represent from 0.5% to 90% of the total weight of the composition. They may be chosen from the group consisting of hydrophilic organic solvents, amphiphilic organic solvents and lipophilic organic solvents, and mixtures thereof.

Among the hydrophilic organic solvents that may be mentioned, for example, are linear or branched lower monoalcohols containing from 1 to 8 carbon atoms, for instance ethanol, propanol, butanol, isopropanol or isobutanol; acetone; polyethylene glycols containing from 6 to 80 ethyleneoxy units; polyols such as propylene glycol, butylene glycol, glycerol or sorbitol; mono- or dialkylisosorbides, the alkyl groups of which contain from 1 to 5 carbon atoms, for instance dimethylisosorbide; glycol ethers, for instance diethylene glycol monomethyl ether or monoethyl ether and propylene glycol ethers, for instance dipropylene glycol methyl ether.

Amphiphilic organic solvents that may be mentioned include polyols such as polypropylene glycol (PPG) derivatives, for instance fatty acid esters of polypropylene glycol and fatty alkyl ethers of PPG, for example PPG-36 oleate and PPG-23 oleyl ether.

Examples of lipophilic organic solvents that may be mentioned include hydrocarbons such as hexane, heptane and octane; monocarboxylic or polycarboxylic acid esters such as diisopropyl adipate, dioctyl adipate, alkyl benzoates and dioctyl malate.

The cosmetic composition according to the invention may also comprise one or more adjuvants chosen from the conventional adjuvants used in cosmetics such as, for example, fillers, pigments, colorants, surfactants, sunscreens, natural or synthetic waxes, antioxidants, fragrances, preserving agents, sequestering agents, agents for preventing hair loss, antidandruff agents, foam stabilizers, propellants, ceramides, vitamins or provitamins, or other well-known cosmetic adjuvants.

A person skilled in the art will take care to select the optional additive(s) and the amount thereof such that the advantageous properties intrinsically associated with the composition of the invention are not adversely affected by the envisaged addition.

The compositions according to the invention may be in any form that is suitable and known to those skilled in the art especially in the form of solutions of the lotion or serum type; in the form of aqueous or aqueous-alcoholic gels; in the form of emulsions obtained by dispersing a fatty phase in an aqueous phase (O/W) or conversely (W/O), of more or less thick liquid consistency, such as more or less unctuous milks and creams. These compositions are prepared according to the usual methods.

The compositions according to the invention are preferably used as hair products, especially for holding the hairstyle or for shaping the hair. They may also give the hair temporary colouring effects, or make protect the hair against the effects of UV radiation, while at the same time providing hair holding or fixing properties.

The hair compositions according to the invention are preferably shampoos, hairsetting gels or lotions, blow-drying lotions, and fixing and styling compositions such as lacquers or sprays.

The lotions may be packaged in various forms, especially in vaporisers, pump-dispenser bottles or in aerosol containers in order to apply the composition in vaporised form or in the form of a mousse. Such packaging forms are indicated, for example, when it is desired to obtain a spray or a mousse for fixing or treating the hair.

The treatment process according to the invention consists in applying the composition to rinsed or unrinsed hair, preferably in the form of a spray, either using a pump-dispenser bottle or using an aerosol.

According to one embodiment of this invention, the compositions are preferably used in leave-in mode.

The hair-shaping process according to the invention consists in spraying a composition according to the invention over the entire head of hair, and leaving the composition to act and to dry. The hair may then be placed in the desired shape, either before the application or immediately afterward.

The drying time may be variable and depends on the nature of the composition.

After combing, the hair has increased sheen and a very pleasant feel.

The examples that follow are intended to illustrate the present invention without being limiting in nature.

### EXAMPLE 1

Composition 1

A polystyrene/poly(ethyl 2-perfluorooctylacrylate) diblock polymer is prepared from the following reagents:

| | |
|---|---|
| monomer 2: ethyl 2-perfluorooctylacrylate | 50 g |
| (Tg = 40° C.) | |
| initiator: ethyl 2-bromoisobutyrate | 0.195 g |
| (EtOCOC (CH₃)₂Br (M = 195 g/mol) | |
| CuBr (M = 143.5 g/mol) | 0.1425 g |
| (amount corresponding to 1 × 10⁻³ mol) | |
| Ligand: bipyridine (M = 156 g/mol) | 0.3125 g |
| (amount corresponding to 2 × 10⁻³ mol) | |

The refractive indices, as measured according to the method indicated above, for polystyrene and for poly(ethyl 2-perfluorooctylacrylate) are, respectively, 1.59 and 1.35. The difference between the two refractive indices is much greater than 0.1.

First Step: Preparation of the Polystyrene

The monomers are distilled beforehand.

The initiator, the copper complex and the ligand are mixed together in a hermetic reactor comprising a nitrogen inlet, followed by addition of monomer 1.

The mixture is heated to 120° C. under nitrogen and then left to react at 120° C. for 4 hours while closing off the nitrogen inlet.

Second Step: Formation of the Second Block at the End of the Polystyrene

Monomer 2 is then added and the mixture is left to react for a further 4 hours at 120° C.

After reaction, the reaction mixture is allowed to cool. A viscous green solution is obtained, which is dissolved in dichloromethane. The polymer solution is passed through neutral alumina and the clear solution obtained is precipitated from a methanol/water (80/20) mixture with a polymer/precipitating agent ratio of 1/5.

96 g of polymer are obtained in the form of a viscous product, which corresponds to a yield of 96%.

The product is characterized by gel permeation chromatography (GPC) in tetrahydrofuran (THF), with one equivalent of linear polystyrene. The detection is performed by light scattering. The block copolymer has a weight-average molar mass of 115 000 g/mol and the polydispersity index is equal to 1.6.

The copolymer thus obtained is soluble in ethyl acetate and composition 1 is prepared by introducing into ethyl acetate 5% by weight of the block copolymer obtained, relative to the total weight of the composition.

Composition 1 is applied at a rate of 1 g per lock of 2.5 g of natural European chestnut hair.

The hair is left to dry in the open air and a markedly improved sheen, compared with the untreated hair, is noted.

The hair is soft and feels volumized.

The locks of treated hair are tested by a panel and the results are collated in Table 1 below.

### COMPARATIVE EXAMPLE 1

Composition A

The polystyrene/poly(ethylene/butylene) block polymer available from the company Shell under the trade name Kraton G1701 is used. This copolymer comprises 37% styrene and 63% ethylene/butylene.

The polystyrene has a refractive index of 1.59, whereas the poly(ethylene/butylene) has a refractive index of 1.5.

Composition A is prepared by introducing 5% by weight of the polymer into ethyl acetate, relative to the total weight of the composition.

Composition A is applied at a rate of 1 g per lock of 2.5 g of natural European chestnut hair.

The locks of treated hair are tested by a panel and the results are collated in Table 1 below.

### COMPARATIVE EXAMPLE 2

Composition B

Composition B is prepared from the following components:

| | | |
|---|---|---|
| Phenylsilicone | | 0.1% |
| (Dow Corning 556 fluid cosmetic) | | |
| Alcohol | qs | 100% |

This composition is then applied at a rate of 1 g per lock of 2.5 g of natural European chestnut hair.

The locks of treated hair are tested by a panel and the results are collated in Table 1 below.

A panel of 10 individuals grades the cosmetic characteristics of the treated and untreated locks:

feel quality (from 0 to 5, 0=very poor, 5=very good),

softness (from 0 to 5, 0=very coarse, 5=very good),

cleanliness of the fingers after touching (from 0 to 5, 0=very poor, 5=very clean),

sheen (from 0 to 5, 0=dull, 5=very shiny),

appearance of the sheen (from 0 to 5, 0=dirty appearance, 5=perfectly natural appearance).

The results are indicated in Table 1 below.

TABLE 1

| | | | Cleanliness | | Appearance |
|---|---|---|---|---|---|
| | Feel | | of the | | of the |
| Locks | quality | Softness | fingers | Sheen | sheen |
| treated with | 4.25 | 3.5 | 4.25 | 4.5 | 4.0 |
| composition 1 | | | | | |
| treated with | 2.25 | 1.75 | 2.0 | 3.5 | 3.0 |
| composition a | | | | | |
| treated with | 1.25 | 2.25 | 1.75 | 4.0 | 0.75 |
| composition B | | | | | |
| untreated | 4 | 3.25 | 5 | 2.75 | 4 |

From the results given in Table 1, the locks treated with composition 1 have cosmetic characteristics that are markedly superior to those of the locks treated with compositions A and B.

In particular, for the locks treated with composition B, the hairs stick together. They may be separated by combing, but the sticky effect of the hair reappears very quickly, giving the hair a dirty appearance.

In contrast, the hair treated with composition 1 does not show this phenomenon at all. The hairs have no tendency to stick together. A markedly improved sheen should also be noted for composition 1, compared with the untreated hair. The hair treated with composition 1 is also soft and feels volumized.

1. A cosmetic composition comprising, in a cosmetically acceptable medium, at least one block copolymer, characterized in that said block copolymer consists of at least two polymer blocks A and B, each of the polymer blocks A having a refractive index that is at least 0.1 unit greater or less than the refractive index of the neighbouring polymer block(s) B.

2. The cosmetic composition as claimed in the preceding claim, characterized in that the number of blocks present in the block copolymer is greater than or equal to 2.

3. The cosmetic composition as claimed in either of the preceding claims, characterized in that the number of blocks present in the block copolymer is equal to 2 or 3.

4. The cosmetic composition as claimed in any one of the preceding claims, characterized in that the blocks are prepared from monomers chosen from acrylic acid, methacrylic acid, N,N-dimethyl-acrylamide, quaternized or unquaternized dimethylaminoethyl methacrylate, methacrylamide, N-t-butylacrylamide, maleic acid and its hemiesters, maleic anhydride, crotonic acid, itaconic acid, acrylamide, hydroxylated (meth)acrylates such as hydroxyethyl methacrylate, diallyldimethylammonium chloride, vinyl-pyrrolidone, vinyl ethers, maleimides, vinylpyridine, vinylimidazole, other polar heterocyclic vinyl compounds, styrenesulfonates, allylic alcohols, vinyl alcohols, salts of any of the acids and amines listed above, acrylic or methacrylic acid esters of C₁-C₁₃ alcohols, fluoroacrylates; styrene, a polystyrene, vinyl acetate, vinyl chloride, vinylidene chloride, vinyl propionate, α-methylstyrene, t-butylstyrene, butadiene, cyclohexadiene, ethylene, propylene, vinyltoluene, and mixtures thereof.

5. The cosmetic composition as claimed in any one of the preceding claims, characterized in that said block copolymer is a polystryene/poly(ethyl 2-perfluorooctylacrylate) copolymer.

6. The cosmetic composition as claimed in any one of the preceding claims, characterized in that said block polymer has a weight-average molar mass, measured by light scattering, of between 10 000 and 500 000 g/mol.

7. The cosmetic composition as claimed in claim 8, characterized in that said block polymer has a weight-average molar mass of between 20 000 and 200 000 g/mol.

8. The cosmetic composition as claimed in any one of the preceding claims, characterized in that the proportion of block copolymer is between 0.001% and 10% by weight relative to the total weight of the cosmetic composition.

9. The cosmetic composition as claimed in any one of the preceding claims, characterized in that said block copolymer is soluble or dispersed in the cosmetically acceptable medium.

10. The cosmetic composition as claimed in claim 9, characterized in that said cosmetically acceptable medium comprises water and/or cosmetically acceptable organic solvents.

11. The cosmetic composition as claimed in claim 10, characterized in that the cosmetically acceptable organic solvents are chosen from hydrophilic organic solvents, amphiphilic organic solvents and lipophilic organic solvents, and mixtures thereof.

12. The cosmetic composition as claimed in claim 11, characterized in that the hydrophilic organic solvents are chosen from linear or branched lower alcohols contaiing from 1 to 8 carbon atoms, acetone, polyethylene glycols containing from 6 to 80 ethyleneoxy units, polyols, mono- or dialkylisosorbides, the alkyl groups of which contain from 1 to 5 carbon atoms, and glycol ethers.

13. The cosmetic composition as claimed in claim 11, characterized in that the amphiphilic organic solvents are chosen from polypropylene glycol (PPG) derivatives, for instance fatty acid esters of polypropylene glycol and fatty alkyl ethers of PPG.

14. The cosmetic composition as claimed in claim 11, characterized in that the lipophilic organic solvents are chosen from hydrocarbons and monocarboxylic or polycarboxylic acid esters.

15. The cosmetic composition as claimed in any one of the preceding claims, characterized in that it is in the form of a hair product.

16. The cosmetic composition as claimed in claim 15, characterized in that it allows shaping of the hair and hold of the hairstyle.

17. A hair treatment process, characterized in that at least one composition as claimed in any one of claims 1 to 16 is applied to the hair.

18. A hair shaping process, characterized in that at least one composition as claimed in any one of claims 1 to 16 is sprayed onto the hair and, after leaving it to act and to dry, the hair is placed in the desired shape.

19. The use of a composition as claimed in any one of claims 1 to 16, as a styling product.
